(19) United States
(12) Patent Application Publication
(10) Pub. No.: US 2022/0333161 A1
(43) Pub. Date: Oct. 20, 2022

(54) METHODS FOR PREDICTING THE RISK OF PROGRESSION AND PHARMACOLOGICAL RESPONSE OF A HUMAN SUBJECT SUFFERING FROM RELAPSING-REMITTING MULTIPLE SCLEROSIS

(71) Applicant: BIONOU RESEARCH S.L., San Juan de Alicante (ES)

(72) Inventor: Vicente Manuel NAVARRO LÓPEZ, Alicante (ES)

(21) Appl. No.: 17/429,906

(22) PCT Filed: Feb. 11, 2020
(86) PCT No.: PCT/EP2020/053479

§ 371 Date: Aug. 10, 2021

(30) Foreign Application Priority Data
Feb. 11, 2019 (EP) 19382093.3

(51) Int. Cl. C12Q 1/689

(52) U.S. Cl. CPC C12Q 1/689; C12Q 1/6851

(57) ABSTRACT

The present invention relates to a biomarker or to a combination of biomarkers in stool samples which on one hand help in the diagnosis of multiple sclerosis, and on the other hand predict the progression of multiple sclerosis, as well as the response to treatment of a subject suffering from this disease.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is SEQUENCE_LISTING_150181_401USPC. The text file is 12.2 KB, was created on Mar. 11, 2022, and is being submitted electronically via EFS-Web.

### BACKGROUND

### Technical Field

The present invention relates to a biomarker or to a combination of biomarkers in intestinal or stool samples which on one hand help in the diagnosis of multiple sclerosis, and on the other hand predict the progression of multiple sclerosis, as well as the response to treatment of a subject suffering from this disease.

### Description of the Related Art

Multiple sclerosis (MS) is a chronic neuroinflammatory disease that affects more than 2 million people worldwide, mainly women. Although it can occur at any age, it is usually diagnosed between 25 and 30 years of age, representing the second cause of disability in young adults. The prevalence of the disease is highest in the northern and southern hemispheres and lowest at the equator, with Europe, northern United States, southern Australia, and New Zealand being the geographical areas with the highest prevalence. In Spain, it is estimated that there are 46,000 people diagnosed with MS and 5,000 in the Autonomous Community of Valencia. Etiology of the disease is unknown though it is believed that the combination of a genetic predisposition and exposure to an environmental factor may trigger the activation of an immune response against the nervous system (NS) mediated by myelin-specific autoreactive T-CD4+ lymphocytes. The external agent may be of an infectious type (e.g., Epstein-Barr virus), although other factors such as smoking, low vitamin D levels, and obesity in adolescence have been studied. Relapsing-remitting multiple sclerosis (RRMS) is the most common clinical form of multiple sclerosis. In relapsing-remitting multiple sclerosis, symptoms appear in acute episodes called “flare-ups” alternating with periods of stability. These flare-ups can leave behind significant physical consequences. The symptoms comprise sensory disorders (tingling), optic neuritis, diplopia, spasticity, coordination disorders, and other dysfunctions. Pharmacological treatments have significantly evolved, yet none of them is capable of curing the disease. Corticoids are the most frequently used in managing flare-ups, while immunomodulatory medicaments are used to slow down their progression.

As a result of the development of advanced identification techniques in molecular microbiology, virtually the entire intestinal microbial community, its functionality, and its metabolic capacities can be studied today. As a consequence, various lines of research aimed at understanding the role of human microbiome in health have emerged in the last decade. Specifically, in the field of demyelinating pathologies, there are various publications linking gut microbiota to the onset of experimental autoimmune encephalomyelitis (EAE) in mice. With regard to its relationship with MS, scientific evidence is still scarce, although some studies with patients diagnosed with RRMS and their comparison with healthy controls are already available. In general, all these studies reported intestinal dysbiosis in subjects with RRMS. Cantarel et al. (USA, 2014) analyzed the intestinal microbial diversity of 7 patients with RRMS and 8 healthy controls to measure the effect of vitamin D supplementation, with an increase in the abundance of Faecalibacterium (which is attributed anti-inflammatory effects due to its role in the production of butyrate) being observed after the intake of this vitamin. In a study conducted by Jangi et al. (Jangi S, Gandhi R, Cox L M, Li N, von Glehn F, Yan R, Patel B, Mazzola M A, Liu S, Glanz B L, et al. Alterations of the human gut microbiome in multiple sclerosis. Nat Commun. 2016 Jun. 28; 7: 12015. doi: 10.1038/ncomms12015), the existence of differences at the genus level in patients with RRMS was verified when comparing samples from 60 patients with those from 43 healthy patients, in addition to establishing relationships with their serological profiles. There was an increase in Methanobrevibacter and Akkermansia, as well as a decrease in Butyricimonas and Prevotella in subjects with RRMS. Chen et al. (USA, 2016) studied samples from 31 patients with RRMS and 36 healthy individuals, with differences in the abundance of 35 taxa being observed in the four main phyla, highlighting the decrease in Prevotella and the Bacteroidetes phylum in patients with RRMS. There are other studies such as the one conducted by Tremlett et al. (Tremlett H, Fadrosh D W, Faruqi A A, et al., Gut microbiota in early pediatric multiple sclerosis: a case-control study. Eur J Neurol, 2016 August; 23 (8): 1308-1321, doi: 10.1111/Jan.13026. Epub 2016 May 13. PubMed PMID: 27176462; PubMed Central PMCID: PMC4955679) in pediatric patients with RRMS, which indicates a relationship between the risk of the onset of flare-ups and the absence of Fusobacteria. The methodology used in all of them was the sequencing of the 16S rRNA gene, with the amplification of the V4 region or the V3-5 region.

### BRIEF SUMMARY

[symbol]

### BRIEF DESCRIPTION OF THE SEVERAL VIEWS OF THE DRAWINGS

FIG. 1. Dependence of the Shannon index for both the group of healthy individuals (control) and patients with RRMS. Species biodiversity in the control group is greater than in the treatment group.

FIGS. 2a and 2b. Comparison of the microbiota composition data by family and genus between the group of healthy individuals and patients with RRMS. The bars represent the mean values of the genus with statistically significant differences (p<0.05) between the two population groups.

FIG. 3. Comparative analysis of the main microbiota components in the healthy population and in patients with RRMS. Group samples and comparisons based on the variability of the bacterial composition at the genus taxonomic level for each patient with RRMS and the healthy population.

FIG. 4. Receiver operating characteristic curves (ROC curves) for calculating area under the curve and cut-off points for sensitivity and specificity for differentiating between case (patient with multiple sclerosis) and control (healthy subject) in those microorganisms in which a significant association is observed at the genus level. The values of significance, cut-off values, and the area under the curve are shown in each figure.

FIG. 5. Diet record of patients with RRMS and healthy controls. Percentage of the main components of the diet, represented as the intake frequency, recorded in the group of patients with active RRMS included in the study.

FIG. 6. Data of patients with Massive stool sample genome sequencing analysis. For each patient, the number of detected Ezakiella sequences is indicated and in brackets the number of outbreaks the patient had during the study.

FIG. 7. Data of patients with Massive stool sample genome sequencing analysis. For each patient, the number of detected Romboutsia sequences is indicated and in brackets the number of outbreaks the patient had during the study.

FIG. 8. Data of patients with Massive stool sample genome sequencing analysis. For each patient, the number of detected Porphyromonas sequences is indicated and in brackets the number of outbreaks the patient had during the study.

FIG. 9. Data of patients with Massive stool sample genome sequencing analysis. For each patient, the number of detected Bilophila sequences is indicated and in brackets the number of outbreaks the patient had during the study.

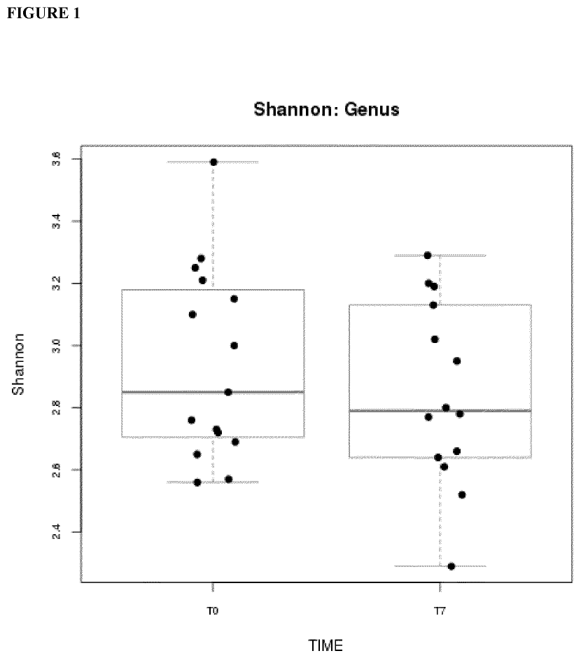

### DETAILED DESCRIPTION

It has been found in the present invention that some of the components of gut microbiota composition are capable of exhibiting differences between healthy subjects without multiple sclerosis and subjects with multiple sclerosis, and furthermore, some of these components are specifically correlated with the progression of multiple sclerosis or its response to treatment.

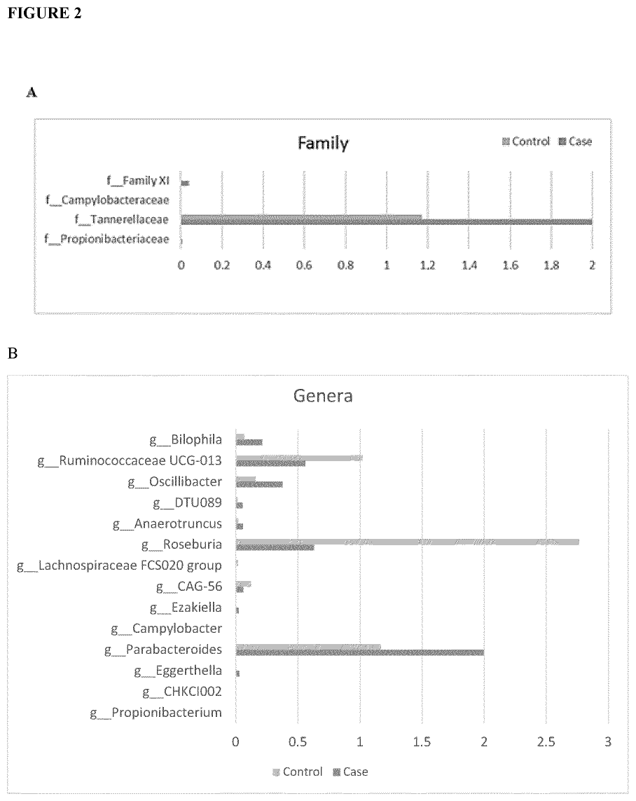

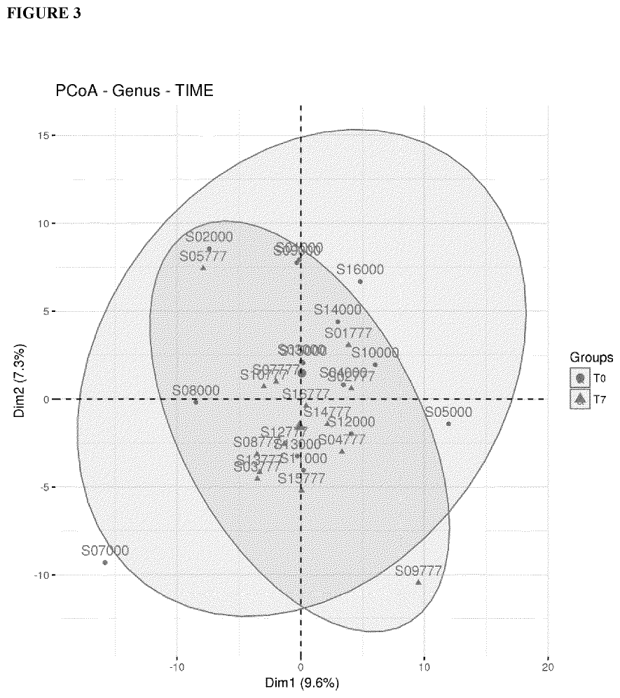

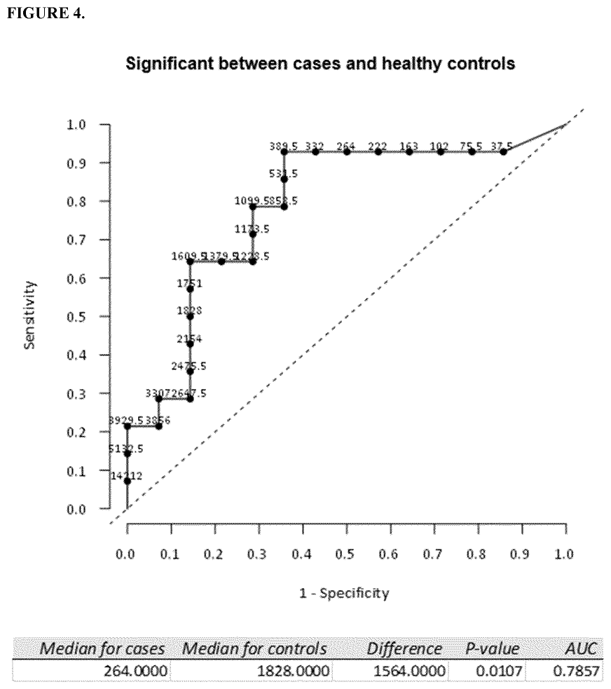

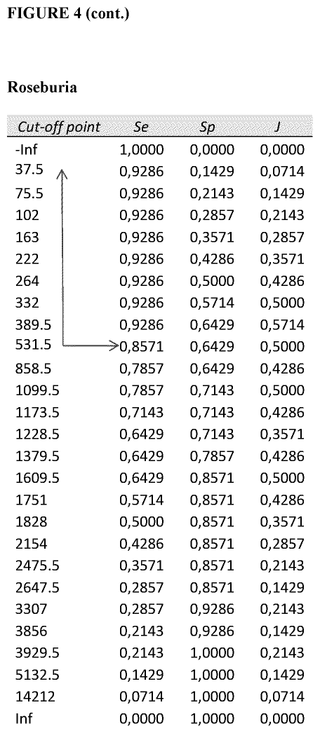

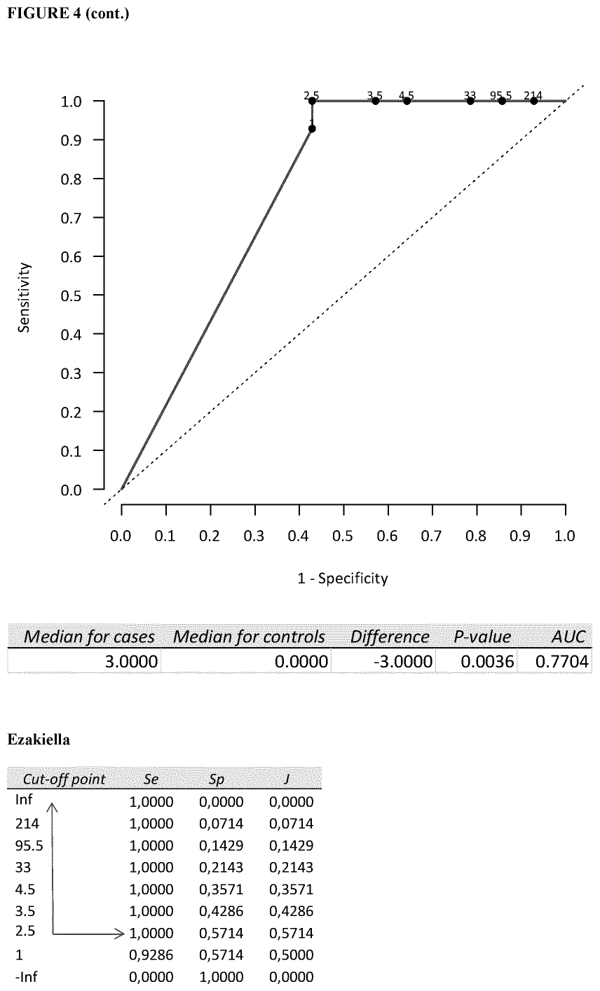

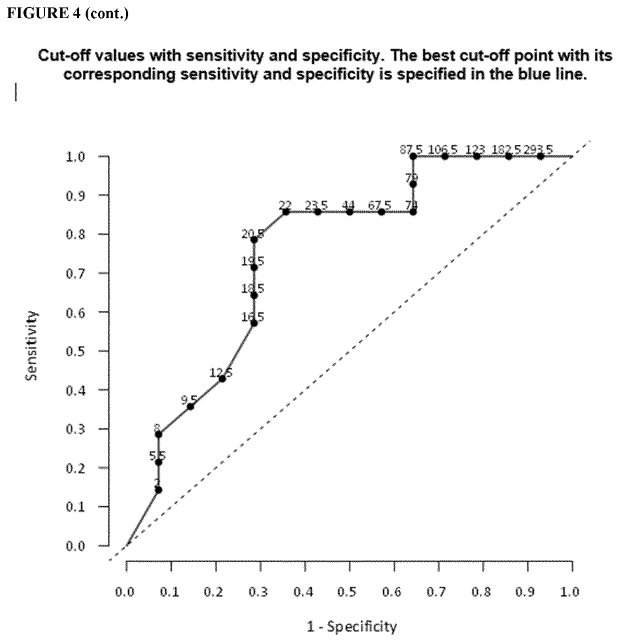

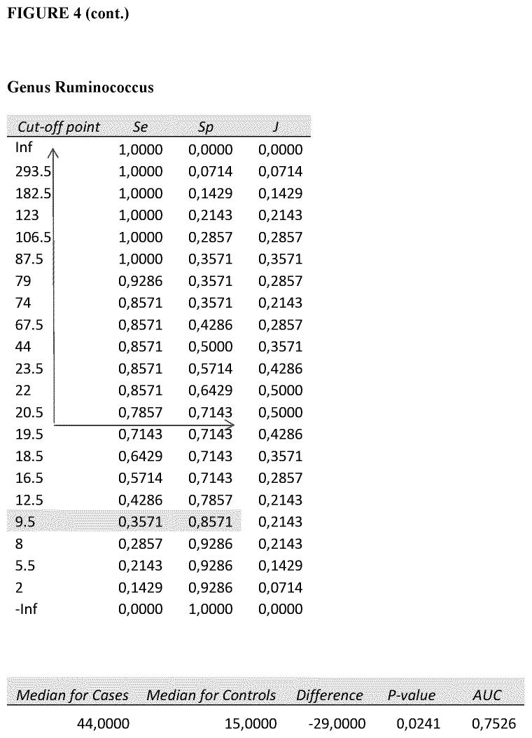

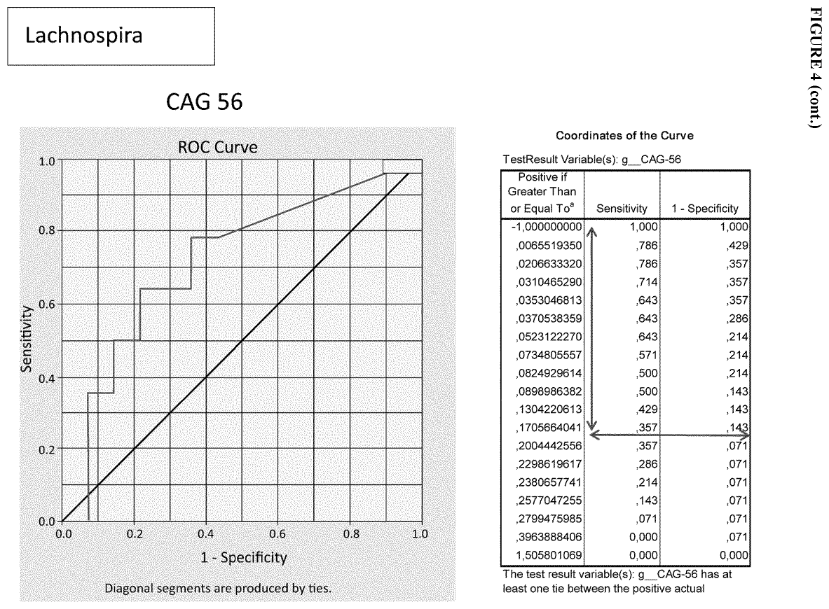

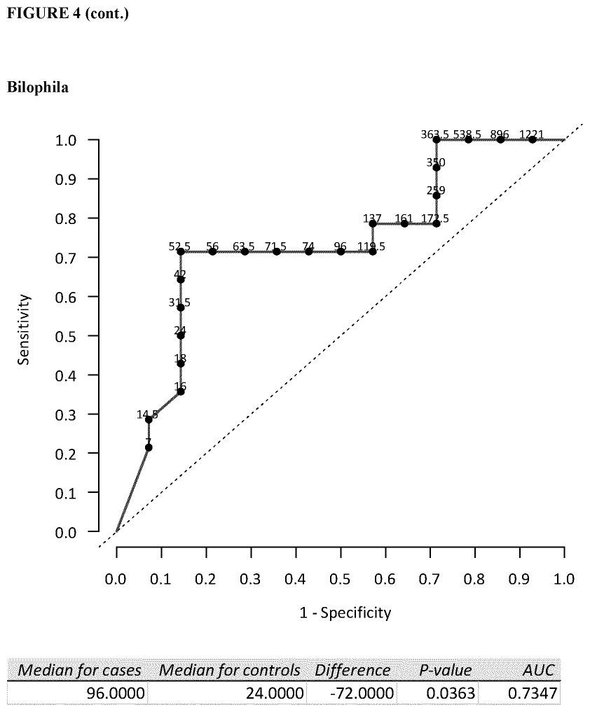

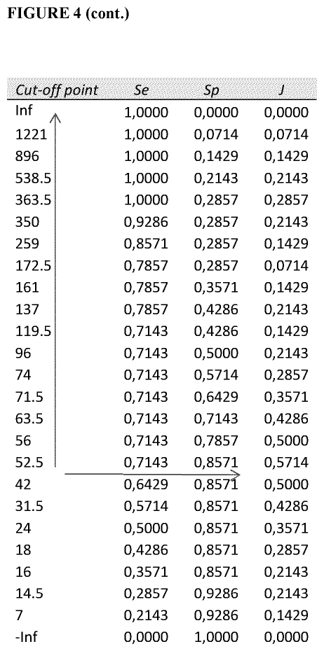

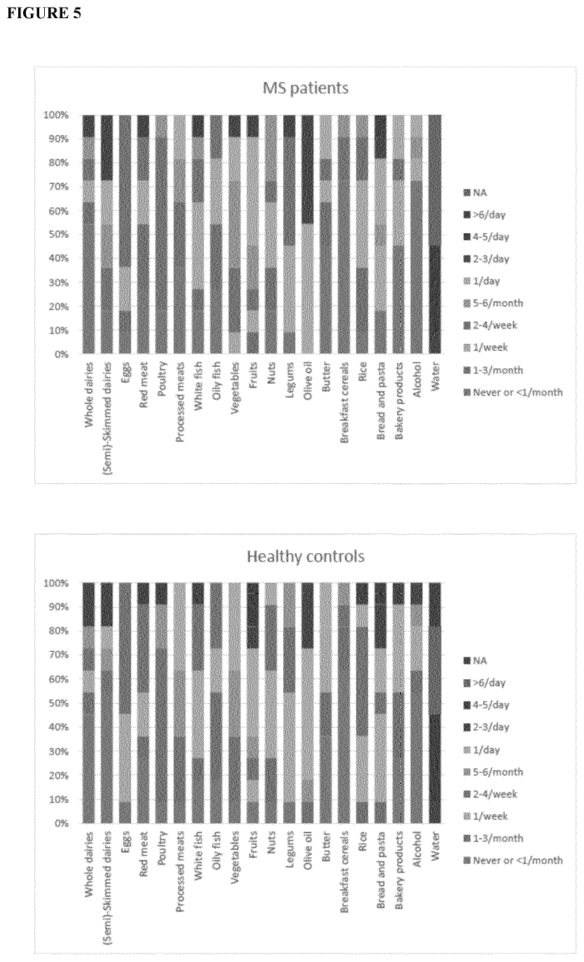

In this sense, as illustrated in the examples of the present invention the authors of the present invention have verified the existence of differences at the genus level of certain microorganisms in patients with RRMS when comparing samples from said patients with those from healthy individuals (see FIG. 2b). Moreover, it was subsequently verified that not all of said genera of microorganisms for which differences between patients and healthy individuals have been determined necessarily provided information which would allow the prognosis of the progression of the course of the disease (multiple sclerosis), i.e., information which would allow predicting the number of relapses in a subject suffering from said disease or determining whether or not said subject was responding to treatment.

Nevertheless, a part of these components identified in intestinal or stool samples from those patients, that were furthermore capable of exhibiting differences between healthy subjects without multiple sclerosis and subjects with multiple sclerosis, were specifically correlated with the progression of multiple sclerosis or its response to treatment. In summary, as shown in detail in the examples and drawings of the present invention, in order to arrive at the results of the present invention the authors investigated the gut microbiota composition in a cohort of 15 individuals with RRMS using 16s rRNA sequencing. Data from the partners of the patients was used as control group because they shared the same diet and biases that diet have on the gut microbial composition were thereby prevented as demonstrated when analyzing the components of the diet and as reflected in the drawings section herein. It must be noted that the microbiota of patients with RRMS differs from the healthy population. In fact, the present invention shows the existence of a significant change in the biodiversity and composition of microorganisms in stool of these patients with respect to the control group consisting of healthy subjects.

In a second phase of the study and once it has been proven that gut microbiota is partially different between a patient with multiple sclerosis and a healthy subject, follow-up was conducted on the group of patients with multiple sclerosis over time. Furthermore, after a follow-up period for the treated patients with multiple sclerosis of at least 3 years, a correlation was observed between certain aspects of the microbiota and the onset of new flare-ups and/or new lesions in the central nervous system (CNS). In this sense, it was observed that patients undergoing treatment with a worse clinical progression and worse response to treatment exhibited microbiota that is different from the microbiota of patients with MS who did respond to treatment. Curiously, those microorganisms that conferred a worse prognosis were furthermore present among those microorganisms found at different concentrations between healthy and sick populations. In other words, these germs seem to be involved in the development or onset of multiple sclerosis, and furthermore, part of them would be involved in a worse progression of the disease despite proper treatment.

In that sense and as illustrated in the examples of the present invention, significant differences were found when comparing cases of multiple sclerosis and healthy controls at the levels of bacteria belonging to the genera Ezakiella, Lachnospira, Bilophila, Ruminococcus, and Roseburia. Moreover, when analyzing only cases of multiple sclerosis and the correlation of onset of new flare-ups with the different microorganisms, it is found that when comparing the levels of bacteria of the genus Ezakiella, there was a positive correlation between the poor progression of the disease and the levels or the concentrations of those bacteria belonging to this genus. Specifically, it was found that for every 100 units of increase in the sequences of Ezakiella, the increase in the risk of flare-ups went up to 1.56 in comparison with patients with zero flare-ups.

Therefore, on one hand these microorganisms appear in different proportions in patients at the time of the diagnosis of multiple sclerosis, and on the other hand a worse prognosis of the disease with a poor response to treatment is observed when these microorganisms are altered with respect to the general population (specifically when there are higher levels of Ezakiella). All this is indicative of the role of the microbiota composition as a factor for predicting the progression of the disease and as an adjunctive test for the diagnosis of multiple sclerosis.

Once this has been done and using the selected germs, a final study of the ROC curve and the area under the curve was performed to define a cut-off value or point for each specific germ, including sensitivity and specificity, which would be the value for predicting whether or not the subject has multiple sclerosis. This data is shown in the drawings and examples.

It can be concluded based on this information that those patients diagnosed with MS have microbiota that is different from the general population despite sharing the same diet. Specifically, the genera Ezakiella, Lachnospira, Bilophila, Ruminococcus, and Roseburia are at different concentrations in patients with MS compared to the general population and this data may be used as a diagnostic test to more preferably be used together with tests already existing for the diagnosis of this pathology. Secondly, it can also be concluded that patients undergoing treatment with a worse clinical progression and worse response to treatment exhibit microbiota different from the microbiota of patients with MS who do respond to treatment, specifically and in this case they exhibit differences in the levels of at least the bacteria belonging to the genus Ezakiella.

It must be noted that in the context of the present invention, the genus Roseburia is found at lower levels in biological intestinal or stool samples in patients with MS compared to the general population. Furthermore, it must be noted that the following sequences SEQ ID NO 1 to SEQ ID NO 3 are sequences representative of this genus and are therefore useful for the identification of bacteria belonging to this genus in said samples through a PCR method or by means of any sequencing technique.

>>bcfaebf36a7617af86ae9f48670ad25b
SEQ ID NO 1
GGGAATATTGCACAATGGGGGAAACCCTGATGCAGCGACGCCGCGTGAGC
GAAGAAGTATTTCGGTATGTAAAGCTCTATCAGCAGGGAAGAAGAATGAC
GGTACCTGACTAAGAAGCACCGGCTAAATACGTGCCAGCAGCCGCGGTAAT
ACGTATGGTGCAAGCGTTATCCGGATTTACTGGGTGTAAAGGGAGCGCAGG
CGGTACGGCAAGTCTGATGTGAAATCCCGGGGCTCAACCCCGGTACTGCAT
TGGAAACTGTCGGACTAGAGTGTCGGAGGGGTAAGTGGAATTCCTAGTGTA
GCGGTGAAATGCGTAGATATTAGGAGGAACACCAGTGGCGAAGGCGGCTT
ACTGGACGATTACTGACGCTGAGGCTCGAAAGCGTGGGGAGCAAAC
>62ea8104bf71ebaa6f22b6a761e62854
SEQ ID NO 2
GGGAATATTGCACAATGGGGGAAACCCTGATGCAGCGACGCCGCGTGAGC
GAAGAAGTATTTCGGTATGTAAAGCTCTATCAGCAGGGAAGAAAAAAATG
ACGGTACCTGACTAAGAAGCCCCGGCTAACTACGTGCCAGCAGCCGCGGTA
ATACGTAGGGGGCAAGCGTTATCCGGATTTACTGGGTGTAAAGGGAGCGCA
GGCGGTGCGGCAAGTCTGATGTGAAAGCCCGGGGCTCAACCCCGGGACTG
CATTGGAAACTGTCGTACTTGAGTATCGGAGAGGTAAGTGGAATTCCTAGT
GTAGCGGTGAAATGCGTAGATATTAGGAGGAACACCAGTGGCGAAGGCGG
CTTACTGGACGATAACTGACGCTGAGGCTCGAAAGCGTGGGGAGCAAAC
>>62df74476957d3c919c8c93fb44ef348
SEQ ID NO 3
GGGAATATTGCACAATGGGGGAAACCCTGATGCAGCGACGCCGCGTGAGC
GAAGAAGTATTTCGGTATGTAAAGCTCTATCAGCAGGGAAGAAGAAATGA
CGGTACCTGACTAAGAAGCACCGGCTAAATACGTGCCAGCAGCCGCGGTA
ATACGTATGGTGCAAGCGTTATCCGGATTTACTGGGTGTAAAGGGAGCGCA
GGCGGAAGGCTAAGTCTGATGTGAAAGCCCGGGGCTCAACCCCGGTACTGC
ATTGGAAACTGGTCATCTAGAGTGTCGGAGGGGTAAGTGGAATTCCTAGTG
TAGCGGTGAAATGCGTAGATATTAGGAGGAACACCAGTGGCGAAGGCGGC
TTACTGGACGATAACTGACGCTGAGGCTCGAAAGCGTGGGGAGCAAAC

One skilled in the art will have knowledge of other sequences representative of this genus which will be used for identifying those bacteria belonging to this genus in a stool or intestinal sample.

Moreover, it must be noted that in the context of the present invention the genus Lachnospira is found at lower levels in biological intestinal or stool samples in patients with MS compared to the general population. Furthermore, it must be noted that the following sequences SEQ ID NO 4 to SEQ ID NO 6 are sequences representative of this genus and are therefore useful for the identification of bacteria belonging to this genus in said samples through a PCR method or by means of any sequencing technique.

>a3723443610f87b1804c73e2aa29abb0
SEQ ID NO 4
GGGAATATTGCACAATGGGGGAAACCCTGATGCAGCGACGCCGCGTGAGC
GAAGAAGTATTTCGGTATGTAAAGCTCTATCAGCAGGGAAGAAAATGACG
GTACCTGACTAAGAAGCCCCGGCTAACTACGTGCCAGCAGCCGCGGTAATA
CGTAGGGGGCAAGCGTTATCCGGATTTACTGGGTGTAAAGGGAGCGTAGAC
GGCGAGGCAAGTCTGATGTGAAAACCCGGGGCTCAACCCCGTGACTGCATT
GGAAACTGTTTTGCTTGAGTGCCGGAGAGGTAAGCGGAATTCCTAGTGTAG
CGGTGAAATGCGTAGATATTAGGAGGAACACCAGTGGCGAAGGCGGCTTA
CTGGACGGCAACTGACGTTGAGGCTCGAAAGCGTGGGGAGCAAAC
>9440de0d588a51ea934e136883c61aac
SEQ ID NO 5
GGGAATATTGCACAATGGGGGAAACCCTGATGCAGCGACGCCGCGTGAGC
GAAGAAGTATTTCGGTATGTAAAGCTCTATCAGCAGGGAAGAAAATGACG
GTACCTGAATAAGAAGCCCCGGCTAACTACGTGCCAGCAGCCGCGGTAATA
CGTAGGGGGCAAGCGTTATCCGGATTTACTGGGTGTAAAGGGAGCGTAGAC
GGCGAGGCAAGTCTGATGTGAAAACCCGGGGCTCAACCCCGTGACTGCATT
GGAAACTGTTTTGCTTGAGTGCCGGAGAGGTAAGCGGAATTCCTAGTGTAG
CGGTGAAATGCGTAGATATTAGGAGGAACACCAGTGGCGAAGGCGGCTTA
CTGGACGGCAACTGACGTTGAGGCTCGAAAGCGTGGGGAGCAAAC
>ea80a53b5eda714de0e6f1f533dcdbdd
SEQ ID NO 6
GGGAATATTGCACAATGGGGGAAACCCTGATGCAGCGACGCCGCGTGAGC
GAAGAAGTATTTCGGTATGTAAAGCTCTATCAGCAGGGAAGAAAATGACG
GTACCTGAATAAGAAGCCCCGGCTAACTACGTGCCAGCAGCCGCGGTAATA
CGTAGGGGGCAAGCGTTATCCGGATTTACTGGGTGTAAAGGGAGCGTAGAC
GGCGAGGCAAGTCTGATGTGAAAGCCCGGGGCTCAACCCCGTGACTGCATT
GGAAACTGTTTTGCTTGAGTGCCGGAGAGGTAAGCGGAATTCCTAGTGTAG
CGGTGAAATGCGTAGATATTAGGAGGAACACCAGTGGCGAAGGCGGCTTA
CTGGACGGCAACTGACGTTGAGGCTCGAAAGCGTGGGGAGCAAAC

Furthermore, it must be noted that in the context of the present invention, the genus Ezakiella is found at higher levels in biological intestinal or stool samples in patients with MS with respect to the general population. Furthermore, it must be noted that the following sequences SEQ ID NO 7 to SEQ ID NO 9 are sequences representative of this genus and are therefore useful for the identification of bacteria belonging to this genus in said samples through a PCR method or by means of any sequencing technique.

>b90dde667311a3c4c3d8bc49cded2d69
SEQ ID NO 7
GGGAATATTGCACAATGGGGGAAACCCTGATGCAGCGACGCCGCGTGAGC
GATGAAGGCTTTCGAGTCGTAAAGCTCTGTCCTATGGGAAGATAATGACGG
TACCATAGAAGAAAGCCCCGGCTAATTACGTGCCAGCAGCCGCGGTAATAC
GTAAGGGGCGAGCGTTGTCCGGAATTATTGGGCGTAAAGAGTGCGTAGGCG
GCAAATTAAGTCAGATGTGAAAACTAAGGGCTCAACCCATAGATTGCATCT
GAAACTGATATGCTTGAGTCAAGGAGAGGAAAGTGGAATTCCTAGTGTAGC
GGTGGAATGCGTAGATATTAGGAGGAATACCGGTGGCGAAGGCGACTTTCT
GGACTTGAACTGACGCTGAGGCACGAAAGCGTGGGGAGCAAAC
>670e10e4a4802959dbb90a7dbc991f22
SEQ ID NO 8
GGGAATATTGCACAATGGAGGGAACTCTGATGCAGCGACGCCGCGTGAGT
GATGAAGGAATTCGTTTCGTAAAACTCTGTTCTATGGGAAGAAAAGGACTG
TACCATAGGAGAAAGCTCCGGCTAAATACGTGCCAGCAGCCGCGGTAATAC
GTATGGAGCGAGCGTTGTCCGGAATTATTGGGCGTAAAGGGTGCGCAGGCG
GCTTTACAAGTTGGATGTGAAATATTGTGGCTCAACCACAAACGTGCATCC
AAAACTGCAAAGCTTGAGTTAAGGAGAGGTAAGTGGAATTCCTGGTGTAGC
GGTGGAATGCGTAGATATCAGGAGGAATACCGGTGGCGAAGGCGACTTAC
TGGACTTAAACTGACGCTCAGGCACGAAAGCGTGGGGAGCAAAC
>737f245bd31abba4f81243d008fd9e34
SEQ ID NO 9
GGGAATATTGCACAATGGAGGGAACTCTGATGCAGCGACGCCGCGTGAGT
GATGAAGGAATTCGTTTCGTAAAACTCTGTTCTATGGGAAGAAAAGGACTG
TACCATAGGAGAAAGCTCCGGCTAAATACGTGCCAGCAGCCGCGGTAATAC
GTATGGAGCGAGCGTTGTCCGGAATTATTGGGCGTAAAGGGTGCGCAGGCG
GCTTTACAAGTTGGATGTGAAATATTGTGGCTCAACCACAAACGTGCATCC
AAAACTGCAAAGCTTGAGTTAAGGAGAGGTAAGTGGAATTCCTGGTGTAGC
GGTGGAATGCGTAGATATCAGGAGGAATACCGGTGGCGAAGGCGACTTAC
TGGACTTAAACTGACGCTCAGGCACGAAAGCGTGGGGAGCAAACA

One skilled in the art will have knowledge of other sequences representative of this genus which will be used for identifying those bacteria belonging to this genus in a stool or intestinal sample.

Finally, it must be noted that in the context of the present invention, the genus Bilophila is found at higher levels in biological intestinal or stool samples in patients with MS with respect to the general population. Furthermore, it must be noted that the following sequences SEQ ID NO 10 to SEQ ID NO 12 are sequences representative of this genus and are therefore useful for the identification of bacteria belonging to this genus in said samples through a PCR method or by means of any sequencing technique.

>8f8744e700fb76316b7af104f71ac34e
SEQ ID NO 10
GGGAATATTGCGCAATGGGCGAAAGCCTGACGCAGCGACGCCGCGTGAGG
GATGAAGGTTCTCGGATCGTAAACCTCTGTCAGGGGGGAAGAAACCCCCTC
GTGTGAATAATGCGAGGGCTTGACGGTACCCCCAAAGGAAGCACCGGCTA
ACTCCGTGCCAGCAGCCGCGGTAATACGGAGGGTGCAAGCGTTAATCGGA
ATCACTGGGCGTAAAGCGCACGTAGGCGGCTTGGTAAGTCAGGGGTGAAAT
CCCACAGCCCAACTGTGGAACTGCCTTTGATACTGCCAGGCTTGAGTACCG
GAGAGGGTGGCGGAATTCCAGGTGTAGGAGTGAAATCCGTAGATATCTGG
AGGAACACCGGTGGCGAAGGCGGCCACCTGGACGGTAACTGACGCTGAGG
TGCGAAAGCGTGGGTAGCAAAC
>dad0fb8c339eb1499fb4e4d1e6b974c2
SEQ ID NO 11
GGGAATATTGCGCAATGGGCGAAAGCCTGACGCAGCGACGCCGCGTGAGG
GATGAAGGTTCTCGGATCGTAAACCCCTGTCAGGGGGGAAGAAACCCCCTC
GTGTGAATAATGCGAGGGCTTGACGGTACCCCCAAAGGAAGCACCGGCTA
ACTCCGTGCCAGCAGCCGCGGTAATACGGAGGGTGCAAGCGTTAATCGGA
ATCACTGGGCGTAAAGCGCACGTAGGCGGCTTGGTAAGTCAGGGGTGAAAT
CCCACAGCCCAACTGTGGAACTGCCTTTGATACTGCCAGGCTTGAGTACCG
GAGAGGGTGGCGGAATTCCAGGTGTAGGAGTGAAATCCGTAGATATCTGG
AGGAACACCGGTGGCGAAGGCGGCCACCTGGACGGTAACTGACGCTGAGG
TGCGAAAGCGTGGGTAGCAAAC
>fca339d0dd6b1dec5d76f2a96dfe0182
SEQ ID NO 12
GGGAATATTGCGCAATGGGCGAAAGCCTGACGCAGCGACGCCGCGTGAGG
GATGAAGGTTCTCGGATCGTAAACCTCTGTCAGGGGGGAAGAAACCCCCTC
GTGTGAATAATGCGAGGGCTTGACGGTACCCCCAAAGGAAGCACCGGCTA
ACTCCGTGCCAGCAGCCGCGGTAATACGGAGGGTGCAAGCGTTAATCGGA
ATCACTGGGCGTAAAGCGCACGTAGGCGGCTTGGTAAGTCAGGGGTGAAAT
CCCACAGCCCAACTGTGGAACTGCCTTTGATACTGTCAGGCTTGAGTACCG
GAGAGGGTGGCGGAATTCCAGGTGTAGGAGTGAAATCCGTAGATATCTGG
AGGAACACCGGTGGCGAAGGCGGCCACCTGGACGGTAACTGACGCTGAGG
TGCGAAAGCGTGGGTAGCAAAC

One skilled in the art will have knowledge of other sequences representative of this genus which will be used for identifying those bacteria belonging to this genus in a stool or intestinal sample.

It must be noted that in the context of the present invention, the genus Ruminococcus is found at higher levels in biological intestinal or stool samples in patients with MS compared to the general population. Furthermore, it must be noted that the following sequences SEQ ID NO 13 to SEQ ID NO 15 are sequences representative of this genus and are therefore useful for the identification of bacteria belonging to this genus in said samples through a PCR method or by means of any sequencing technique.

>ca7d260498fc98f5b1203d3a5bf0cba0
SEQ ID NO 13
GGGAATATTGCGCAATGGAGGAAACTCTGACGCAGTGACGCCGCGTATAG
GAAGAAGGTTTTCGGATTGTAAACTATTGTCGTTAGGGAAGATACAAGACA
GTACCTAAGGAGGAAGCTCCGGCTAACTACGTGCCAGCAGCCGCGGTAATA
CGTAGGGAGCAAGCGTTATCCGGATTTATTGGGTGTAAAGGGTGCGTAGAC
GGGACAACAAGTTAGTTGTGAAATCCCTCGGCTTAACTGAGGAACTGCAAC
TAAAACTATTGTTCTTGAGTGTTGGAGAGGAAAGTGGAATTCCTAGTGTAG
CGGTGAAATGCGTAGATATTAGGAGGAACACCGGTGGCGAAGGCGACTTTC
TGGACAATAACTGACGTTGAGGCACGAAAGTGTGGGGAGCAAAC
>4142cb390a45ceac09f3fef2846b7d22
SEQ ID NO 14
GGGAATATTGGACAATGGAGGAAACTCTGATCCAGTGACGCCGCGTGAAG
GAAGAAGGTCTTCGGATTGTAAACTTATTTTATCAGGGAAGAAGAAAGTGA
CAGTACCTGAAGAAAAAGGACCGGCAAACTACGTGCCAGCAGCCGCGGTA
ATACGTAGGGTCCAAGCGTTATCCGGATTTACTGGGTGTAAAGGGCGAGTA
GACGGCGCTGTAAGTCAGCTGTGAAAACTTAGGGCTCAACCTTAAGCCTGC
AGCTGAAACTGTAGTGCTAGAGTGCAGGAGAGGTAAGCGGAATTCCTAGTG
TAGCGGTGAAATGCGTAGATATTAGGAGGAACACCAGTGGCGAAGGCGGC
TTACTGGACTGTAACTGACGTTGAGGCGCGAAAGTGTGGGGAGCAAAC
>471de9965b88e25338c306b99ecede17
SEQ ID NO 15
GGGAATATTGCGCAATGGAGGAAACTCTGACGCAGTGACGCCGCGTGCAG
GAAGAAGGTTTTCGGATTGTAAACTGCTTTTAACAGGAAAGAAAAAAATGA
CGGTACCTGTTGAATAAGCTCCGGCTAACTACGTGCCAGCAGCCGCGGTAA
TACGTAGGGAGCGAGCGTTATCCGGATTTATTGGGTGTAAAGGGTGCGTAG
ACGGAAATGCAAGTTAGTTGTGAAATACCTCGGCTTAACTGAGGAACTGCA
ACTAAAACTATATTTCTTGAGTATCGGAGGGGTTTGTGGAATTCCTAGTGT
AGCGGTGAAATGCGTAGATATTAGGAAGAACACCGGTGGCGAAGGCGACAA
ACTGGACGATAACTGACGTTGAGGCACGAAAGTGTGGGGAGCAAAC

One skilled in the art will have knowledge of other sequences representative of this genus which will be used for identifying those bacteria belonging to this genus in a stool or intestinal sample.

The following Table 1 summarizes the diagnostic information indicated above.

TABLE 1

| Genus | Correlation | Value | Sensitivity | Specificity |
|---|---|---|---|---|
| Roseburia | − | 390 | 0.92 | 0.63 |
| Lachnospira | − | 120 | 0.35 | 0.86 |
| Ezakiella | + | 2.5 | 1 | 0.57 |
| Bilophila | + | 52 | 0.71 | 0.85 |
| Ruminococcus | + | 22 | 0.86 | 0.65 |

Therefore, a first aspect of the present invention relates to the in vitro use of the level or the concentration in an intestinal or stool sample of bacteria belonging to the genus Ezakiella, Lachnospira, Bilophila, Ruminococcus, or Roseburia, or any combination thereof, for the diagnosis/of multiple sclerosis in a patient, or for obtaining useful data which allows said diagnosis.

According to the data shown in the examples, for diagnosis, Roseburia with a 390 cut-off point, 92% sensitivity, and 63% specificity would be the best indicator of the presence of MS individually. If all the indicators (Roseburia, Ezakiella, Bilophila, and Lachnospira, and optionally Ruminococcus) were combined, sensitivity would increase to 100% and Ezakiella with a 2.5 cut-off point (57% specificity and 100% sensitivity), Bilophila with a 52 cut-off point (85% specificity and 71% sensitivity), and Lachnospira with a 120 cut-off point (86% specificity and 35% sensitivity) could be used.

An alternative embodiment of the first aspect of the invention relates to a method for the in vitro diagnosis or for obtaining useful data which helps in said diagnosis of a subject suspected of possibly suffering from multiple sclerosis, which method comprises using, as an indicator in an intestinal or stool sample obtained from said subject, the level or the concentration in said sample of bacteria belonging to the genus Ezakiella, Lachnospira, Bilophila, Ruminococcus, or Roseburia, or any combination thereof, wherein if the level or the concentration in said intestinal or stool sample of bacteria belonging to Ezakiella, Lachnospira, Bilophila, Ruminococcus, or Roseburia, or any combination thereof differs or varies from that present in an intestinal or stool sample obtained from a healthy subject or with respect to a reference value, it is indicative of said subject having multiple sclerosis.

In a preferred embodiment of the first aspect of the invention, the method comprises using, as an indicator in an intestinal or stool sample obtained from said subject, the level or the concentration in said sample of bacteria belonging to the genus Bilophila, wherein if the level or the concentration in said intestinal or stool sample of bacteria belonging to said genus is increased with respect to that present in an intestinal or stool sample obtained from a healthy subject or with respect to a reference value, it is indicative of said subject having multiple sclerosis.

In another preferred embodiment of the first aspect of the invention, the method comprises using, as an indicator in an intestinal or stool sample obtained from said subject, of the level or the concentration in said sample of bacteria belonging to the genus Ruminococcus, wherein if the level or the concentration in said intestinal or stool sample of bacteria belonging to said genus is increased with respect to that present in an intestinal or stool sample obtained from a healthy subject or with respect to a reference value, it is indicative of said subject having multiple sclerosis.

In another preferred embodiment of the first aspect of the invention, the method comprises using, as an indicator in an intestinal or stool sample obtained from said subject, the level or the concentration in said sample of bacteria belonging to the genus Ezakiella, wherein if the level or the concentration in said intestinal or stool sample of bacteria belonging to said genus is increased with respect to that present in an intestinal or stool sample obtained from a healthy subject or with respect to a reference value, it is indicative of said subject having multiple sclerosis.

In another preferred embodiment of the first aspect of the invention, the method comprises using, as an indicator in an intestinal or stool sample obtained from said subject, the level or the concentration in said sample of bacteria belonging to the genus Lachnospira, wherein if the level or the concentration in said intestinal or stool sample of bacteria belonging to said genus is decreased with respect to that present in an intestinal or stool sample obtained from a healthy subject or with respect to a reference value, it is indicative of said subject having multiple sclerosis.

In another preferred embodiment of the first aspect of the invention, the method comprises using, as an indicator in an intestinal or stool sample obtained from said subject, the level or the concentration in said sample of bacteria belonging to the genus Roseburia, wherein if the level or the concentration in said intestinal or stool sample of bacteria belonging to said genus is decreased with respect to that present in an intestinal or stool sample obtained from a healthy subject or with respect to a reference value, it is indicative of said subject having multiple sclerosis.

In yet another preferred embodiment of the first aspect of the invention, the method comprises using, as an indicator in an intestinal or stool sample obtained from said subject, the level or the concentration in said sample of bacteria belonging to the genera Ezakiella, Lachnospira, Bilophila, and Roseburia, and optionally Ruminococcus, wherein if the level or the concentration in said intestinal or stool sample of bacteria belonging to the genera Roseburia and Lachnospira is decreased with respect to that present in an intestinal or stool sample obtained from a healthy subject or with respect to a reference value, and wherein if the level or the concentration in said intestinal or stool sample of bacteria belonging to the genera Ezakiella, Ruminococcus, and Bilophila is increased with respect to that present in an intestinal or stool sample obtained from a healthy subject or with respect to a reference value, it is indicative of said subject having multiple sclerosis.

In the context of the present invention, multiple sclerosis is understood to be a progressive disease of the central nervous system causing multiple lesions in the myelin covering axons of neurons and constituting the white substance, in the form of disseminated plaques; and RRMS is understood to be a predominant type of multiple sclerosis in which symptoms present in the form of flare-ups that can last for days, weeks, and even months, and will vary from one episode to another, according to the affected area of the central nervous system.

It must be noted that the present invention sufficiently describes the representative nucleotide sequences (i.e., SEQ ID No 1 to SEQ No 15) which allow identifying the presence as well as the concentration of each of the genera herein mentioned.

A second aspect of the present invention relates to an in vitro method for the prognosis of the progression of multiple sclerosis in a subject, comprising the following steps:

a. determining the levels or the concentration of bacteria belonging to the genus Ezakiella, Bilophila, Porphyromonas, and/or Romboutsia, or any combination thereof, in an intestinal or stool sample isolated from said subject; and

b. comparing the levels or the concentration of said bacteria in said intestinal or stool sample with the value obtained in a sample from a healthy subject or with a reference value, wherein an increase in the number of sequences of Ezakiella, Bilophila, Porphyromonas, and/or Romboutsia in said sample with respect to the control or the reference value, the increase in the risk of flare-ups goes up, in comparison with patients with zero flare-ups. In other words, an increase in the level or the concentration of bacteria belonging to any of the above-mentioned genus or any combination thereof, in an intestinal or stool sample isolated from said subject with respect to the control, is indicative of a poor progression of the disease.

In a preferred embodiment, the reference value is taken from patients with zero flare-ups.

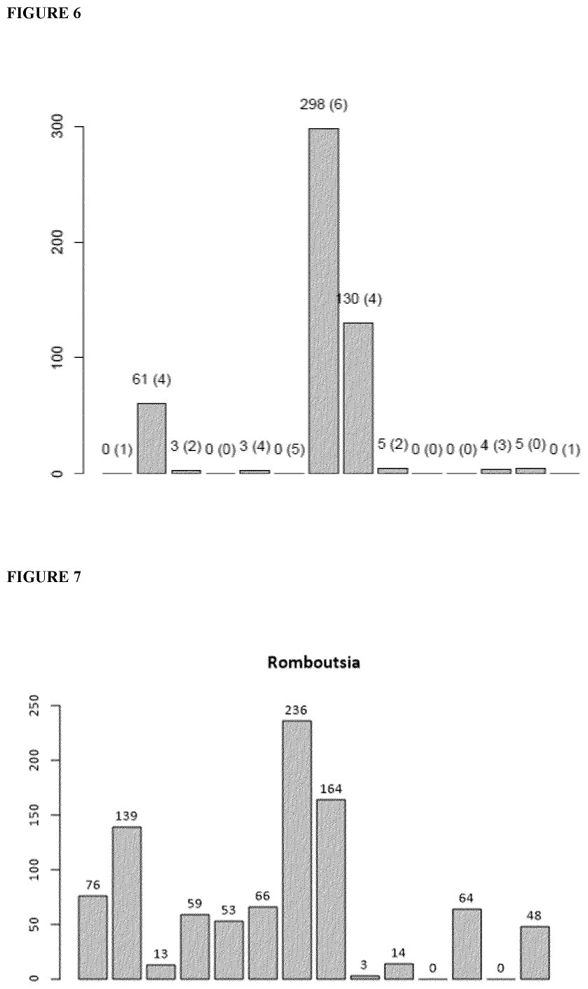

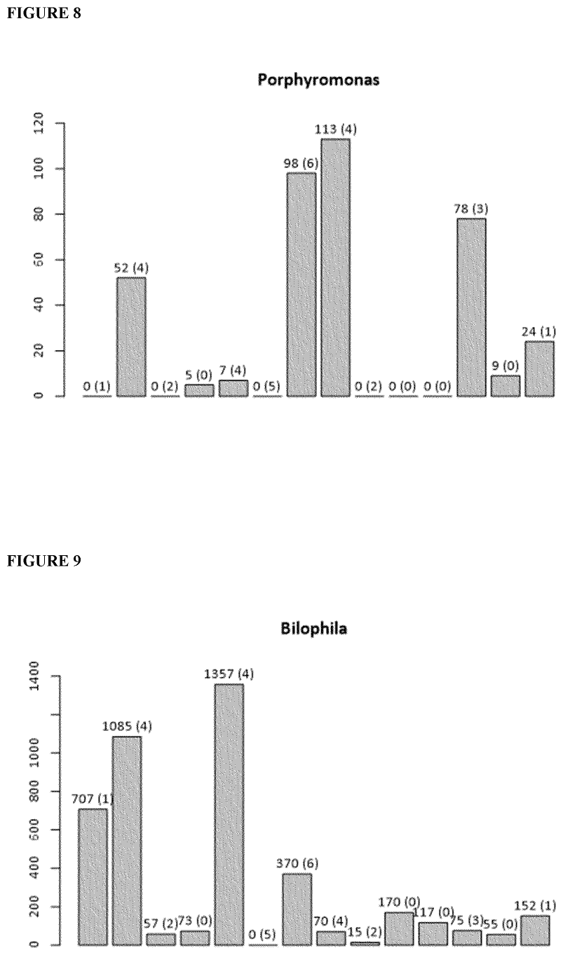

In a preferred embodiment of the second aspect of the invention, and as shown in FIG. 6, for every 100 units of increase in the sequences of Ezakiella in said sample with respect to the control, the increase in the risk of flare-ups goes up preferably by an approximate value of 1.56, in comparison with patients with zero flare-ups. In other words, an increase in the level or the concentration of bacteria belonging to the genus Ezakiella in an intestinal or stool sample isolated from said subject with respect to the control is indicative of a poor progression of the disease. It is noted that the same statistical analysis to establish the relationship between the number of flares (relapses) (flare-ups) and the number of sequences detected for each of the bacteria gender was performed for the rest of the above-mentioned genders. In the case of Romboutsia: for every increase of 100 units in the number of detected sequences of the bacteria, the number of flare-ups was increased by 74%. Analysis in the case of Porphyromonas shows how as for every increase of 50 units in the number of detected sequences of the bacteria, the number of flare-ups was increased by 73%. Finally, analysis of data in the case of Bilophila shows how as for every increase of 300 units in the number of detected sequences of the bacteria, the number of flare-ups was increased by 18% (see example 5 and FIGS. 6 to 9).

In the context of the present invention, poor progression of the disease is understood to be the presence of one or more new flare-ups of the disease regardless of whether or not the multiple sclerosis is being treated. However, preferably in the context of the present invention said prognosis is carried out in subjects treated with any of the drugs prescribed for this disease, preferably with one or more of those drugs identified in Table 2.

In the context of the present invention, reference value is preferably understood to be the result of the data of a mathematical algorithm which uses the concentration and the total amount of each bacterium belonging to one or more of the genera proposed in the present invention in the general population or in a healthy subject. The best sensitivity and specificity value will automatically be proposed using the algorithm. This algorithm will provide values along with the proposed value, with the changing sensitivity and specificity of the values in order to provide physicians with more information and to allow them to decide on the best test and cut-off value for each patient or specific situation.

In the context of the present invention, the bacteria belonging to any of the genera proposed in an intestinal or stool sample isolated from said subject can be determined, in a non-limiting manner, by means of massive sequencing of the genome of the stool such that the total number of sequences of that bacterium existing in the stool is obtained together with the total number of other bacteria present in that stool sample. Preferably, said determination is performed by means of a PCR or real-time PCR method.

In another preferred embodiment of the first or second aspect of the invention or of any of the preferred embodiments of the invention, said levels or concentration of bacteria refer to the total amount of the bacteria belonging to the genus with respect to the total bacteria present in said sample.

In another preferred embodiment of the first or second aspect of the invention or of any of the preferred embodiments of the invention, the multiple sclerosis is relapsing-remitting multiple sclerosis (RRMS).

In another preferred embodiment of the first or second aspect of the invention or of any of the preferred embodiments of the invention, the levels or the concentration of bacteria belonging to the genera Ezakiella, Lachnospira, Bilophila, Porphyromonas, Romboutsia and/or Roseburia are determined by performing an amplification reaction from a nucleic acid preparation derived from said sample using a pair of primers capable of amplifying one or more representative regions of said genera.

In yet another preferred embodiment of the first or second aspect of the invention or of any of the preferred embodiments of the invention, the amplification reaction is carried out by means of a real-time polymerase chain reaction. Preferably, the detection of the amplification product is carried out by means of a fluorescent intercalating agent. Even more preferably, the detection of the amplification product(s) is carried out by means of a labeled probe, wherein the probe preferably comprises at its 5′ end a reporter pigment and at its 3′ end a “quencher” pigment or silencer or buffering agent. Preferably, in the case of Bilophila, the amplification reaction is carried out by using the following primers of SEQ ID NO 16 and 17:

| | | |
|---|---|---|
| Bil1 | F | GGT ATG TTC GAT CCG GCT ATC |
| | R | TGT TTT CCC GAC CAT CTT CC |

A third aspect of the invention relates to an in vitro method for the prediction of the therapeutic response of a patient diagnosed with multiple sclerosis, preferably relapsing-remitting multiple sclerosis, wherein said method comprises the steps of determining and comparing the levels or the concentration of bacteria according to any of the embodiments of the second aspect of the invention, and wherein an increase in the level or the concentration of bacteria belonging to the genus Ezakiella, Bilophila, Porphyromonas, and/or Romboutsia is indicative of a lack of response to treatment, preferably of any of the treatments identified in Table 2.

In a fourth aspect of the invention, the method of the first, second, or third aspect of the invention or of any of the embodiments thereof further comprises storing the results of the method in a data carrier, wherein said data carrier is preferably a computer readable medium.

In a fifth aspect of the invention, the method of the first, second, or third aspect of the invention or of any of the embodiments thereof comprises at least the implementation of the comparative step and optionally the provision of a result as a consequence of said comparison using a computer program.

A sixth aspect of the invention relates to a kit comprising one or more pairs of primers capable of amplifying bacteria belonging to the genera Ezakiella, Lachnospira, Bilophila, Porphyromonas, Romboutsia and/or Roseburia. Preferably and as mentioned, a sixth aspect of the invention relates to a method for the detection of bacteria from a stool or intestinal sample, comprising the following steps:

i) contacting the sample to be analyzed with a reaction mixture containing specific primers capable of amplifying bacteria belonging to the genera Ezakiella, Lachnospira, Bilophila, Porphyromonas, Romboutsia and/or Roseburia preferably for performing multiplex PCR,

ii) performing amplification by means of polymerase chain reaction,

iii) identifying the formation of the products of the preceding step, said formation being indicative of the levels or the concentration of bacteria belonging to one or more of the genera Ezakiella, Lachnospira, Bilophila, and/or Roseburia.

In relation to this sixth aspect of the invention, it preferably provides a method for simultaneously detecting Ezakiella, Lachnospira, Bilophila, and/or Roseburia.

In a particular embodiment of this sixth aspect of the invention, DNA fragments included or comprised in sequences 1 to 12 are amplified.

In another embodiment of this sixth aspect of the invention, the amplification products which allow identifying the different bacterial species and groups are detected by means of using probes. In a more preferred embodiment, these probes have a length between 15 and 25 nucleotides. The primers can be designed by means of multiple alignment with programs such as CLUSTAL X, which allow identifying highly conserved regions that serve as a template. In another particular embodiment of this first

Given the great abundance of PCR inhibitors, such as humic and fulvic acids, heavy metals, heparin, etc., which may give rise to false negatives, and despite the existence of methods which reduce the concentration of molecules of this type, it is advisable (cf. J. Hoorfar et al., “Making internal amplification control mandatory for diagnostic PCR” J. of Clinical Microbiology, December 2003, pp. 5835) for PCR assays to contain an internal amplification control (IAC). This IAC is simply a DNA fragment which is amplified simultaneously with the target sample, such that its absence at the end of the assays is indicative of the presence of factors which have led to an unwanted PCR development.

Throughout the description, the term “specific” means that the primers comprise a nucleotide sequence that is completely complementary to the genes or gene fragments used by the present invention.

A preferred embodiment of the sixth aspect of the invention relates to the use of the kit or of the methodology therein described for implementing the methodology according to any of the first, second, or third aspects of the present invention.

In summary, the knowledge acquired about gut microbiota composition in patients undergoing treatment along with the development of the diagnosis or prognosis KIT based on this gut microbiota is of crucial importance, as it would allow the neurologist treating these patients to attain data about the probability of the patient presenting or not presenting multiple sclerosis during their first visits, as well as the probability of response to treatment by identifying those patients with a worse prognosis.

The following examples merely illustrate the present invention and must not be understood as limiting same.

1-11. (canceled)

12. A analytical method for determining the levels or the concentration of bacteria belonging to the genus Bilophila, Ezakiella, Porphyromonas, and/or Romboutsia, or any combination thereof, in an intestinal or stool sample, comprising
a. performing an amplification reaction from a nucleic acid preparation derived from an intestinal or stool sample isolated from a subject suspected of having relapsing-remitting multiple sclerosis (RRMS) by using at least one pair of primers capable of amplifying at least one representative region of said genus;
b. detecting an amplification product of the amplification reaction; and
c. determining the levels or the concentration of said bacteria in said intestinal or stool sample relative to the level or the concentration of the same bacteria in an intestinal or stool sample isolated from a subject having zero flare-ups.

13. The analytical method according to claim 12, comprising the following steps:
a. contacting the nucleic acid preparation with a reaction mixture containing specific primers capable of amplifying bacteria belonging to the genus Bilophila, Ezakiella, Porphyromonas, and/or Romboutsia,
b. performing the amplification by means of polymerase chain reaction,
c. identifying the formation of the products of the amplification, said formation being indicative of the levels or the concentration of said bacteria.

14. The analytical method according to claim 12, wherein the amplification reaction is carried out by means of a real-time polymerase chain reaction.

15. The analytical method according to claim 12, wherein DNA fragments included or comprised in SEQ ID NOs: 4 to 6 are amplified.

16. The analytical method according to claim 12, wherein the amplification products are detected by means of using probes.

17. The analytical method according to claim 16, wherein the probes have a length between 15 and 25 nucleotides.

18. The analytical method according to claim 16, wherein the amplification products are detected by means of using labeled probes.

19. The analytical method according to claim 12, wherein said method further comprises storing the results of the method in a data carrier,

20. The analytical method according to claim 19, wherein said data carrier is a computer readable medium.

21. A method for treating a patient having multiple sclerosis (MS), comprising:
a. administering to the patient a treatment for MS; and
i. continuing to administer the treatment to the patient determined to have no increase, relative to a reference value, in the level or the concentration of bacteria belonging to the genus Bilophila, Ezakiella, Porphyromonas, and/or Romboutsia in an intestinal or stool sample from the patient; or
ii. discontinuing to administer the treatment to the patient determined to have an increase, relative to the reference value, in said level or the concentration of said bacteria in said sample,
wherein the level or the concentration of said bacteria has been determined by an analytical method comprising:
b. performing an amplification reaction from a nucleic acid preparation derived from the sample by using at least one pair of primers capable of amplifying at least one representative region of said genus;
c. detecting an amplification product of the amplification reaction; and
d. determining the levels or the concentration of said bacteria in said sample relative to the reference value, wherein the reference value is the level or the concentration of the same bacteria in an intestinal or stool sample isolated from a subject having zero flare-ups.

22. The method according to claim 21, wherein said treatment is selected from the group consisting of interferon beta, interferon beta 1a, interferon beta 1b, natalizumab, fingolimod, dimethyl fumarate, teriflunomide, and glatiramer acetate.

23. The method according to claim 21, wherein the analytical method comprises the following steps:
a. contacting the nucleic acid preparation with a reaction mixture containing specific primers capable of amplifying bacteria belonging to the genus Bilophila, Ezakiella, Porphyromonas, and/or Romboutsia,
b. performing the amplification by means of polymerase chain reaction,
c. identifying the formation of the products of the amplification, said formation being indicative of the levels or the concentration of said bacteria.

24. The method according to claim 21, wherein the amplification reaction is carried out by means of a real-time polymerase chain reaction.

25. The method according to claim 21, wherein DNA fragments included or comprised in SEQ ID NOs: 4 to 6 are amplified.

26. The method according to claim 21, wherein the amplification products are detected by means of using probes.

27. The method according to claim 26, wherein the probes have a length between 15 and 25 nucleotides.

28. The method according to claim 26, wherein the amplification products are detected by means of using labeled probes.

29. A kit comprising:
i. primers capable of amplifying bacteria belonging to the genus Bilophila, Ezakiella, Porphyromonas, and/or Romboutsia;
ii. labeled probes capable of identifying amplified bacteria belonging to the genus; and
iii. directions for use of the primers to amplify a nucleic acid preparation derived from an intestinal or stool sample isolated from a subject suspected of having relapsing-remitting multiple sclerosis (RRMS), and determining the levels or the concentration of said bacteria in said intestinal or stool sample relative to the level or the concentration of the same bacteria in an intestinal or stool sample isolated from a subject having zero flare-ups.

30. The kit of claim 29, wherein the probes comprise at their 5′ end a reporter pigment and at their 3′ end a quencher pigment or silencer or buffering agent.

31. The kit of claim 29, wherein the directions comprise amplifying DNA fragments included or comprised in SEQ ID NOs: 4 to 6.


SEQUENCE LISTING (17 sequences)

SEQ ID NO: 1 - DNA, 400 bp, Artificial Sequence
  Roseburia sp.
  bcfaebf36a7617af86ae9f48670ad25b
  misc_feature (1)..(400)
gggaatattgcacaatgggggaaaccctgatgcagcgacgccgcgtgagcgaagaagtatttcggtatgtaaagctctatcagcagggaagaagaatgacggtacctgactaagaagcaccggctaaatacgtgccagcagccgcggtaatacgtatggtgcaagcgttatccggatttactgggtgtaaagggagcgcaggcggtacggcaagtctgatgtgaaatcccggggctcaaccccggtactgcattggaaactgtcggactagagtgtcggaggggtaagtggaattcctagtgtagcggtgaaatgcgtagatattaggaggaacaccagtggcgaaggcggcttactggacgattactgacgctgaggctcgaaagcgtggggagcaaac

SEQ ID NO: 2 - DNA, 402 bp, Artificial Sequence
  Roseburia sp.
  62ea8104bf71ebaa6f22b6a761e62854
  misc_feature (1)..(402)
gggaatattgcacaatgggggaaaccctgatgcagcgacgccgcgtgagcgaagaagtatttcggtatgtaaagctctatcagcagggaagaaaaaaatgacggtacctgactaagaagccccggctaactacgtgccagcagccgcggtaatacgtagggggcaagcgttatccggatttactgggtgtaaagggagcgcaggcggtgcggcaagtctgatgtgaaagcccggggctcaaccccgggactgcattggaaactgtcgtacttgagtatcggagaggtaagtggaattcctagtgtagcggtgaaatgcgtagatattaggaggaacaccagtggcgaaggcggcttactggacgataactgacgctgaggctcgaaagcgtggggagcaaac

SEQ ID NO: 3 - DNA, 401 bp, Artificial Sequence
  Roseburia sp.
  62df74476957d3c919c8c93fb44ef348
  misc_feature (1)..(401)
gggaatattgcacaatgggggaaaccctgatgcagcgacgccgcgtgagcgaagaagtatttcggtatgtaaagctctatcagcagggaagaagaaatgacggtacctgactaagaagcaccggctaaatacgtgccagcagccgcggtaatacgtatggtgcaagcgttatccggatttactgggtgtaaagggagcgcaggcggaaggctaagtctgatgtgaaagcccggggctcaaccccggtactgcattggaaactggtcatctagagtgtcggaggggtaagtggaattcctagtgtagcggtgaaatgcgtagatattaggaggaacaccagtggcgaaggcggcttactggacgataactgacgctgaggctcgaaagcgtggggagcaaac

SEQ ID NO: 4 - DNA, 399 bp, Artificial Sequence
  Lachnospira sp.
  a3723443610f87b1804c73e2aa29abb0
  misc_feature (1)..(399)
gggaatattgcacaatgggggaaaccctgatgcagcgacgccgcgtgagcgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgaggcaagtctgatgtgaaaacccggggctcaaccccgtgactgcattggaaactgttttgcttgagtgccggagaggtaagcggaattcctagtgtagcggtgaaatgcgtagatattaggaggaacaccagtggcgaaggcggcttactggacggcaactgacgttgaggctcgaaagcgtggggagcaaac

SEQ ID NO: 5 - DNA, 399 bp, Artificial Sequence
  Lachnospira sp.
  9440de0d588a51ea934e136883c61aac
  misc_feature (1)..(399)
gggaatattgcacaatgggggaaaccctgatgcagcgacgccgcgtgagcgaagaagtatttcggtatgtaaagctctatcagcagggaagaaaatgacggtacctgaataagaagccccggctaactacgtgccagcagccgcggtaatacgtagggggcaagcgttatccggatttactgggtgtaaagggagcgtagacggcgaggcaagtctgatgtgaaaacccggggctcaaccccgtgactgcattggaaactgttttgcttgagtgccggagaggtaagcggaattcctagtgtagcggtgaaatgcgtagatattaggaggaacaccagtggcgaaggcggcttactggacggcaactgacgttgaggctcgaaagcgtggggagcaaac

SEQ ID NO: 6 - DNA, 399 bp, Artificial Sequence
  Lachnospira sp.
  ea80a53b5eda714de0e6f1f533dcdbdd
  misc_feature (1)..(399)
gggaatattgcacaatgggggaaaccctgatgcagcgacgccgcgtgagcgaagaagtatttcggtatgtaaagctctatcagcagggaagaaaatgacggtacctgaataagaagccccggctaactacgtgccagcagccgcggtaatacgtagggggcaagcgttatccggatttactgggtgtaaagggagcgtagacggcgaggcaagtctgatgtgaaagcccggggctcaaccccgtgactgcattggaaactgttttgcttgagtgccggagaggtaagcggaattcctagtgtagcggtgaaatgcgtagatattaggaggaacaccagtggcgaaggcggcttactggacggcaactgacgttgaggctcgaaagcgtggggagcaaac

SEQ ID NO: 7 - DNA, 399 bp, Artificial Sequence
  Ezakiella sp.
  b90dde667311a3c4c3d8bc49cded2d69
  misc_feature (1)..(399)
gggaatattgcacaatgggggaaaccctgatgcagcgacgccgcgtgagcgatgaaggctttcgagtcgtaaagctctgtcctatgggaagataatgacggtaccatagaagaaagccccggctaattacgtgccagcagccgcggtaatacgtaaggggcgagcgttgtccggaattattgggcgtaaagagtgcgtaggcggcaaattaagtcagatgtgaaaactaagggctcaacccatagattgcatctgaaactgatatgcttgagtcaaggagaggaaagtggaattcctagtgtagcggtggaatgcgtagatattaggaggaataccggtggcgaaggcgactttctggacttgaactgacgctgaggcacgaaagcgtggggagcaaac

SEQ ID NO: 8 - DNA, 399 bp, Artificial Sequence
  Ezakiella sp.
  670e10e4a4802959dbb90a7dbc991f22
  misc_feature (1)..(399)
gggaatattgcacaatggagggaactctgatgcagcgacgccgcgtgagtgatgaaggaattcgtttcgtaaaactctgttctatgggaagaaaaggactgtaccataggagaaagctccggctaaatacgtgccagcagccgcggtaatacgtatggagcgagcgttgtccggaattattgggcgtaaagggtgcgcaggcggctttacaagttggatgtgaaatattgtggctcaaccacaaacgtgcatccaaaactgcaaagcttgagttaaggagaggtaagtggaattcctggtgtagcggtggaatgcgtagatatcaggaggaataccggtggcgaaggcgacttactggacttaaactgacgctcaggcacgaaagcgtggggagcaaac

SEQ ID NO: 9 - DNA, 400 bp, Artificial Sequence
  Ezakiella sp.
  737f245bd31abba4f81243d008fd9e34
  misc_feature (1)..(400)
gggaatattgcacaatggagggaactctgatgcagcgacgccgcgtgagtgatgaaggaattcgtttcgtaaaactctgttctatgggaagaaaaggactgtaccataggagaaagctccggctaaatacgtgccagcagccgcggtaatacgtatggagcgagcgttgtccggaattattgggcgtaaagggtgcgcaggcggctttacaagttggatgtgaaatattgtggctcaaccacaaacgtgcatccaaaactgcaaagcttgagttaaggagaggtaagtggaattcctggtgtagcggtggaatgcgtagatatcaggaggaataccggtggcgaaggcgacttactggacttaaactgacgctcaggcacgaaagcgtggggagcaaaca

SEQ ID NO: 10 - DNA, 425 bp, Artificial Sequence
  Bilophila sp.
  8f8744e700fb76316b7af104f71ac34e
  misc_feature (1)..(425)
gggaatattgcgcaatgggcgaaagcctgacgcagcgacgccgcgtgagggatgaaggttctcggatcgtaaacctctgtcaggggggaagaaaccccctcgtgtgaataatgcgagggcttgacggtacccccaaaggaagcaccggctaactccgtgccagcagccgcggtaatacggagggtgcaagcgttaatcggaatcactgggcgtaaagcgcacgtaggcggcttggtaagtcaggggtgaaatcccacagcccaactgtggaactgcctttgatactgccaggcttgagtaccggagagggtggcggaattccaggtgtaggagtgaaatccgtagatatctggaggaacaccggtggcgaaggcggccacctggacggtaactgacgctgaggtgcgaaagcgtgggtagcaaac

SEQ ID NO: 11 - DNA, 425 bp, Artificial Sequence
  Bilophila sp.
  dad0fb8c339eb1499fb4e4d1e6b974c2
  misc_feature (1)..(425)
gggaatattgcgcaatgggcgaaagcctgacgcagcgacgccgcgtgagggatgaaggttctcggatcgtaaacccctgtcaggggggaagaaaccccctcgtgtgaataatgcgagggcttgacggtacccccaaaggaagcaccggctaactccgtgccagcagccgcggtaatacggagggtgcaagcgttaatcggaatcactgggcgtaaagcgcacgtaggcggcttggtaagtcaggggtgaaatcccacagcccaactgtggaactgcctttgatactgccaggcttgagtaccggagagggtggcggaattccaggtgtaggagtgaaatccgtagatatctggaggaacaccggtggcgaaggcggccacctggacggtaactgacgctgaggtgcgaaagcgtgggtagcaaac

SEQ ID NO: 12 - DNA, 425 bp, Artificial Sequence
  Bilophila sp.
  fca339d0dd6b1dec5d76f2a96dfe0182
  misc_feature (1)..(425)
gggaatattgcgcaatgggcgaaagcctgacgcagcgacgccgcgtgagggatgaaggttctcggatcgtaaacctctgtcaggggggaagaaaccccctcgtgtgaataatgcgagggcttgacggtacccccaaaggaagcaccggctaactccgtgccagcagccgcggtaatacggagggtgcaagcgttaatcggaatcactgggcgtaaagcgcacgtaggcggcttggtaagtcaggggtgaaatcccacagcccaactgtggaactgcctttgatactgtcaggcttgagtaccggagagggtggcggaattccaggtgtaggagtgaaatccgtagatatctggaggaacaccggtggcgaaggcggccacctggacggtaactgacgctgaggtgcgaaagcgtgggtagcaaac

SEQ ID NO: 13 - DNA, 400 bp, Artificial Sequence
  Ruminococcus sp.
  ca7d260498fc98f5b1203d3a5bf0cba0
  misc_feature (1)..(400)
gggaatattgcgcaatggaggaaactctgacgcagtgacgccgcgtataggaagaaggttttcggattgtaaactattgtcgttagggaagatacaagacagtacctaaggaggaagctccggctaactacgtgccagcagccgcggtaatacgtagggagcaagcgttatccggatttattgggtgtaaagggtgcgtagacgggacaacaagttagttgtgaaatccctcggcttaactgaggaactgcaactaaaactattgttcttgagtgttggagaggaaagtggaattcctagtgtagcggtgaaatgcgtagatattaggaggaacaccggtggcgaaggcgactttctggacaataactgacgttgaggcacgaaagtgtggggagcaaac

SEQ ID NO: 14 - DNA, 402 bp, Artificial Sequence
  Ruminococcus sp.
  4142cb390a45ceac09f3fef2846b7d22
  misc_feature (1)..(402)
gggaatattggacaatggaggaaactctgatccagtgacgccgcgtgaaggaagaaggtcttcggattgtaaacttattttatcagggaagaagaaagtgacagtacctgaagaaaaaggaccggcaaactacgtgccagcagccgcggtaatacgtagggtccaagcgttatccggatttactgggtgtaaagggcgagtagacggcgctgtaagtcagctgtgaaaacttagggctcaaccttaagcctgcagctgaaactgtagtgctagagtgcaggagaggtaagcggaattcctagtgtagcggtgaaatgcgtagatattaggaggaacaccagtggcgaaggcggcttactggactgtaactgacgttgaggcgcgaaagtgtggggagcaaac

SEQ ID NO: 15 - DNA, 402 bp, Artificial Sequence
  Ruminococcus sp.
  471de9965b88e25338c306b99ecede17
  misc_feature (1)..(402)
gggaatattgcgcaatggaggaaactctgacgcagtgacgccgcgtgcaggaagaaggttttcggattgtaaactgcttttaacaggaaagaaaaaaatgacggtacctgttgaataagctccggctaactacgtgccagcagccgcggtaatacgtagggagcgagcgttatccggatttattgggtgtaaagggtgcgtagacggaaatgcaagttagttgtgaaatacctcggcttaactgaggaactgcaactaaaactatatttcttgagtatcggaggggtttgtggaattcctagtgtagcggtgaaatgcgtagatattaggaagaacaccggtggcgaaggcgacaaactggacgataactgacgttgaggcacgaaagtgtggggagcaaac

SEQ ID NO: 16 - DNA, 21 bp, Artificial Sequence
  Forward primer Bilophila
  misc_feature (1)..(21)
ggtatgttcgatccggctatc

SEQ ID NO: 17 - DNA, 20 bp, Artificial Sequence
  Reverse primer Bilophila
  misc_feature (1)..(20)
tgttttcccgaccatcttcc